(19) United States
(12) Patent Application Publication
(10) Pub. No.: US 2012/0003274 A1
(43) Pub. Date: Jan. 5, 2012

(54) ORAL SUSTAINED RELEASE ANTIDEPRESSANT FORMULATION

(75) Inventors: Barry Scott Brand, Cary, NC (US); James Cecil Free, Apex, NC (US)

(73) Assignee: CeNeRx Biopharma, Inc., Cary, NC (US)

(21) Appl. No.: 13/143,403

(22) PCT Filed: Jan. 8, 2010
(86) PCT No.: PCT/US2010/020468

§ 371 Date: Sep. 19, 2011

Related U.S. Application Data
(60) Provisional application No. 61/143,767, filed on Jan. 9, 2009.

(51) Int. Cl. A61K 9/00; A61K 31/39; A61P 25/24; C07D 327/08

(52) U.S. Cl. 424/400; 549/16; 514/434

(57) ABSTRACT

Pharmaceutical presentations or phenoxathiin-based MAO-A inhibitors are disclosed whereby the MAO receptors are capable of sustained release in the digestive tract. Particular phenoxathiin-based MAO-A inhibitors include those of the following formula: wherein n is 0, 1 or 2; R¹ is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl. A wide variety or sustained release mechanisms can be utilized so as to provide gradual release of the active ingredient after ingestion as a pharmaceutical presentation, such as a tablet or capsule. Presentations include sustained release tablets, sustained release capsules, capsules containing sustained release beads.

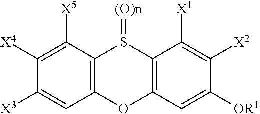

### BACKGROUND

1. Technical Field

Provided herein are oral sustained release pharmaceutical formulations, products and related methods. In particular, provided herein are oral sustained release pharmaceutical formulations, products comprising 3-fluoro-7-(2,2,2-trifluoroethoxy)phenoxathiin 10,10-dioxide as an active ingredient, and related methods.

2. Background

The transient elevation of blood pressure, leading in some cases to hypertensive crisis, has been noted in patients treated with monoamine oxidase inhibitor (MAOI) agents, such as phenelzine, isocarboxazide, ipraniazid, and tranylcypromine following the consumption of tyramine-rich dietary foods and beverages. This acute form of hypertension, similar to that seen in patients with phaeochromocytoma, has been referred to in the medical literature as the “cheese effect” or “cheese reaction” because of the high tyramine content found in some aged cheeses. Because of this potentially dangerous food reaction, physicians have been reluctant to prescribe MAOIs even though they are highly effective in the treatment of major depressive disorder, social phobia and panic attack.

Therefore, there remains a need for suitable MAOIs that do not elicit dangerous food reactions or require strict dietary restrictions. The formulations, products and methods provided herein address this need and provide additional advantages.

### SUMMARY

Sustained release pharmaceutical presentations of phenoxathiin-based MAO-A inhibitors are disclosed whereby the MAO receptors are protected from binding to active ingredient in the stomach. Particular phenoxathiin-based MAO-A inhibitors include those of the following formula:

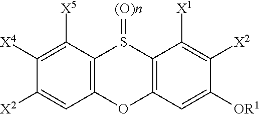

wherein n is 0, 1 or 2; R¹ is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl. Examples of phenoxathiin-based MAO-A inhibitors include, but are not limited to, 3-fluoro-7-(2,2,2-trifluoroethoxy)phenoxathiin 10,10-dioxide (hereinafter “CX157”) of the following formula:

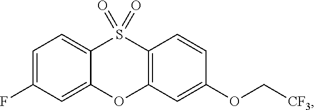

3-(2,2,2-trifluoroethoxy)phenoxathiin 10,10-dioxide (hereinafter “CX009”) of the following formula:

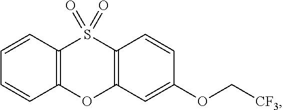

and 3-(2,2,2-trifluoro-1-methylethoxy)phenoxathiin 10,10-dioxide (hereinafter “CX2614”) of the following formula:

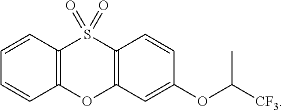

Such presentations do not elicit dangerous food reactions or require strict dietary restrictions by virtue of reducing release of the active ingredient and thereby reducing the degree to which the MAO-A inhibitor blocks MAO receptors from binding dietary tyramine.

In some embodiments, such presentations the product is a tablet or capsule or a core sheathed in an annular body. In some embodiments, such presentations the product is formulated so as to achieve plasma levels of phenoxathiin-based MAO-A inhibitor raging from about 40 ng/ml to about 80 ng/ml. In some embodiments, such presentations the product contains said 3-fluoro-7-(2,2,2-trifluoroethoxy)phenoxathiin 10,10-dioxide as the sole active ingredient. In some embodiments, such presentations the 3-fluoro-7-(2,2,2-trifluoroethoxy)phenoxathiin 10,10-dioxide is characterized as having a melting point at about 169-175° C. In some embodiments, such presentations the 3-fluoro-7-(2,2,2-trifluoroethoxy)phenoxathiin 10,10-dioxide is characterized as being in crystalline form and having an x-ray powder diffraction peak at 2θ=11.0°, using CuKα radiation. In some embodiments, such presentations the product is a tablet containing about 50 to 500 milligrams of 3-fluoro-7-(2,2,2-trifluoroethoxy)phenoxathiin 10,10-dioxide.

Also provided are oral pharmaceutical dosage forms comprising 3-fluoro-7-(2,2,2-trifluoroethoxy)phenoxathiin 10,10-dioxide and adapted to retard release of 3-fluoro-7-(2,2,2-trifluoroethoxy)phenoxathiin 10,10-dioxide in the digestive tract. In some embodiments, such presentations the dosage form is a tablet, a capsule, or a core sheathed in an annular body.

### BRIEF DESCRIPTION OF THE DRAWINGS

FIG. 1 is a diagram of the enzymatic barriers and enzymes involved in the biotransformation of orally administered tyramine in an unmedicated subject (upper portion) and in a MAO-A inhibited subject (lower portion). In humans, the activity of MAO-A and MAO-B is as follows: intestinal mucosa, 90% and 10%: liver, 30% and 70%: adrenergic nerve terminal, 100% and 0%, respectively. Abbreviations: HPAA=p-hydroxyphenylacetic acid; tyramine=free tyramine; Ty-SO4=tyramine sulfate; NA=noradrenaline; Oct.=octopamine; COMT=catachol-O-methyltransferase.

### DETAILED DESCRIPTION

Monoamine oxidase inhibitor (MAOI) agents can cause dangerous food reactions following the consumption of tyramine-rich dietary foods and beverages. This dangerous side-effect has minimized the use of MAOIs even though they are highly effective in the treatment of major depressive disorder, social phobia and panic attack. Reversible inhibitors of monoamine oxidase type-A (RIMAs) are a family of psychiatric drugs and natural compounds that inhibit monoamine oxidase temporarily and reversibly. However, therapeutic doses of some RIMAs can still potentiate the tyramine pressor effect as much as 40- to 50-fold. As a result, RIMAs also are seldom used therapeutically.

As provided herein, greater safety factors for a particular class of RIMAs, termed phenoxathiin-based MAO-A inhibitors (also referred to herein as “active” or “active ingredient”), as defined above in the Summary, can be achieved through an sustained release formulation that avoids peak exposure levels associated with an immediate-release formulations, yet is engineered to deliver a antidepressant amount of drug that reversibly competes with dietary tyramine for MAO-A in the gastrointestinal and hepatic tissues. Such a formulation is particularly effective with CX157 since this RIMA is devoid of inhibitory actions on MAO-B, thus allowing tyramine inactivation through the MAO-B pathway. Thus the specific and reversible properties of CX157 as a MAO-A inhibitor provide a favorable profile for a weak potentiating effect on the oral tyramine pressor effect.

In accordance with the above, various sustained release formulations and presentations of phenoxathiin-based MAO-A inhibitors, and particularly, of CX157 are provided herein. For example, clinical trial and commercial tablets of a phenoxathiin-based MAO-A inhibitor such as CX157 can be coated, encapsulated or otherwise treated so as to render the tablet capable of sustained release.

As used herein, all expressions of percentage, ratio, proportion and the like, will be in weight units unless otherwise stated. Expressions of proportions of the sustained release product will refer to the product in dried form, after the removal of the water in which many of the ingredients are dissolved or dispersed. The term “sugar” refers to a sugar other than a reducing sugar. A reducing sugar is a carbohydrate that reduces Fehling's (or Benedict's) or Tollens' reagent. All monosaccharides are reducing sugars as are most disaccharides with the exception of sucrose. One common binding or filling agent is lactose. This excipient is particularly useful for tablets since it compresses well, is both a diluent and binder, and is cheap. However, it is a reducing sugar and it may be that the active ingredient interacts with lactose both at room temperature and under accelerated stability conditions (heat). Therefore, avoidance of lactose and other reducing sugars from formulations comprising the active ingredient may be important. As discussed below, sucrose is a particular sugar.

In a particular sustained release product, a core of active is surrounded by an sustained release coat and formed into a pellet. The pellets can then be loaded into gelatin capsules. The various components and layers of the pellet will be individually discussed as follows, together with the methods of adding the different ingredients to build up the pellet.

### A. Active Ingredient

Sustained release pharmaceutical presentations of phenoxathiin-based MAO-A inhibitors are provided whereby the binding of MAO receptors in the digestive tract by the phenoxathiin-based MAO-A inhibitors is reduced. Particular active ingredients used in the formulations provided herein are phenoxathiin-based MAO-A inhibitors having the following formula:

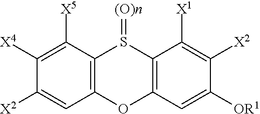

wherein n is 0, 1 or 2; R¹ is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl.

A particular active ingredient of the sustained release pharmaceutical product comprises 3-fluoro-7-(2,2,2-trifluoroethoxy)phenoxathiin 10,10-dioxide (also referred to herein as CX157) of the following formula:

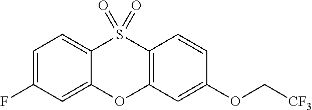

as an active ingredient. Another particular core of the sustained release pharmaceutical product comprises 3-(2,2,2-trifluoroethoxy)phenoxathiin 10,10-dioxide (also referred to herein as CX009) of the following formula:

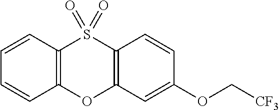

as an active ingredient. Another particular core of the sustained release pharmaceutical product comprises 3-(2,2,2-trifluoro-1-methylethoxy)phenoxathiin 10,10-dioxide (hereinafter “CX2614”) of the following formula:

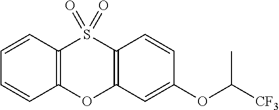

as an active ingredient. Methods for preparation of the above phenoxathiin-based MAO-A inhibitors and other phenoxathiin-based MAO-A inhibitors are known in the art, as exemplified in U.S. Pat. No. 6,110,961, which is incorporated by reference herein in its entirety.

Also provided herein are oral compositions such as tablets or capsules containing said active ingredient which have a low excipient load such that once or twice a day dosing is possible, preferably with one or two such compositions being administered at each dosing. The sustained release product provided herein can utilize any physical form of the active ingredient. When the active pharmaceutical ingredient is CX157, the active ingredient can be in the “high melt” crystalline form.

The “high melt” crystalline form for CX157 is taught in U.S. application Ser. No. 11/773,892, which is incorporated by reference herein in its entirety, where “Form A” of the aforementioned application is the form referred to herein as “high melt.” Briefly, the high melt form can be characterized as having a melting point at about 169-176° C.; about 170-174° C., about 171-173° C., about 171-172° C., or about 171° C. The high melt form is distinguishable from at least one other form of 3-fluoro-7-(2,2,2-trifluoroethoxy)phenoxathiin-10,10-dioxide, which melts at about 158-163° C., typically about 160-162° C. The high melt form also can be characterized as containing less than about 1% H₂O, about 1%-0.001% H₂O, about 0.5%-0.01% H₂O, about 0.05%-0.01% H₂O, or about 0.02% H₂O, as determined by the Karl Fischer method. In addition, the high melt form can be characterized as having an attenuated total reflectance Fourier transform infrared spectrum at 1480-1440 cm⁻¹ substantially identical to FIG. 2(a) of the aforementioned application, having an attenuated total reflectance Fourier transform infrared spectrum at 970-800 cm⁻¹ substantially identical to FIG. 2(a) of the aforementioned application, or having an attenuated total reflectance Fourier transform infrared spectrum substantially identical to FIG. 2(a) of the aforementioned application. The attenuated total reflectance Fourier transform infrared spectrum of the high melt form is distinguishable from the attenuated total reflectance Fourier transform infrared spectrum at 970-800 cm⁻¹ and 1480-1440 cm⁻¹ of another form of CX157, which is substantially identical to FIG. 2(b) of the aforementioned application. The high melt form can further be characterized as dissolving at about 75-85° C., about 75-80° C., about 75-78° C., or about 75-77° C. in a solvent that is 10% (v/v) water in acetic acid when the ratio (w/v) of compound to solvent is about 1.6 g:10 mL.

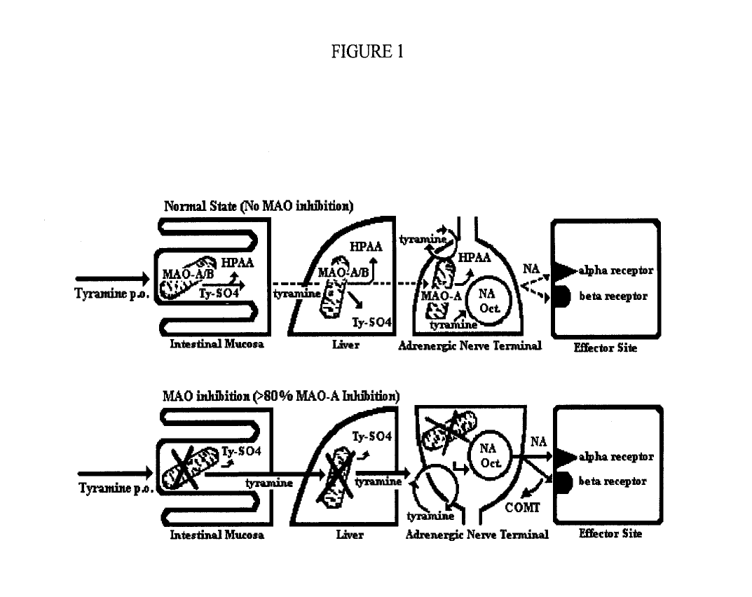

The high melt form can be characterized as having a major x-ray powder diffraction peak at about d spacings 4.0, 4.4 and/or 8.0. The high melt form can be characterized as substantially lacking an x-ray powder diffraction peak at about d spacings 10.3, 7.3, and/or 3.65. The high melt form can be characterized as having a major x-ray powder diffraction peak at about 2θ=11.0°, 20.1°, and/or 22.2°, using CuK_{α} radiation. The high melt form also can be characterized as substantially lacking an x-ray powder diffraction peak at 2θ=8.5°, 12.0°, and/or 24.6°, using CuK_{α} radiation. The high melt form also can be characterized as having an x-ray powder diffraction pattern substantially identical to FIG. 1(a) of the aforementioned application. The x-ray powder diffraction pattern of the high melt form is distinguishable from the x-ray powder diffraction properties of another form of CX157, which has major peaks at about d spacings 10.3, 7.3, and/or 3.65, and about 2θ=11.0°, 20.1°, and/or 22.2°, using CuK₆₀ radiation, and has an x-ray powder diffraction pattern substantially identical to FIG. 1(b) of the aforementioned application.

### B. Sustained Release Formulations

Sustained-release pharmaceutical formulations can be configured in a variety of dosage forms, such as tablets and beads; can contain a variety of fillers and excipients, such as retardant excipients (also referred to a release modifiers); and can be made in a variety of ways. Those skilled in the art can determine the appropriate configuration by routine experimentation guided by the descriptions provided herein.

Sustained-release pharmaceutical formulations can contain fillers. Examples of suitable fillers include, but are not limited to, METHOCEL® methylcellulose, hydroxypropyl methylcellulose (HPMC), hydroxypropylcellulose (HPC), corn starch, polyvinyl alcohol (PVA), polyvinylpyrrolidone (PVP), cross-linked PVP, and the like.

Sustained-release pharmaceutical formulations can contain excipients. Examples of suitable excipients include, but are not limited to, acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypryopl cellulose (L-HPC), magnesium stearate, maltodextrin, maltose, mannitol, methylcellulose, microcrystalline cellulose, methacrylate, sodium carboxymethylcellulose, polyvinyl acetate phthalate (PVAP), povidone, shellac, sodium starch glycolate, sorbitol, starch, sucrose, triacetin, triethylcitrate, vegetable based fatty acid, xanthan gum, xylitol, and the like.

In preferred embodiments, the sustained-release pharmaceutical formulation comprises active ingredient methylcellulose and microcrystalline cellulose. In some embodiments, the formulation comprises, for example, from about 30%, 40%, or 50%, to about 80% or 90% active ingredient by weight. In some embodiments, the formulation comprises about 0.1%, 0.5%, 1%, 3%, 5%, 10% or 20% active ingredient by weight. Preferably, the active ingredient is present at a percentage of about 55%, 60%, 65%, or 70% by weight. In other preferred embodiments, the formulation comprises about 95% active ingredient.

The active ingredient can be present in at least, or at least about, more than, or more than about, 10 mg, 15 mg, 20 mg, 25 mg, 30 mg, 40 mg, 50 mg, 60 mg, 70 mg, 80 mg, 90 mg, 100 mg, 125 mg, 150 mg, 175 mg, or 200 mg. The active ingredient can be present in up to, or up to about, less than, or less than about, 25 mg, 30 mg, 40 mg, 50 mg, 60 mg, 70 mg, 80 mg, 90 mg, 100 mg, 125 mg, 150 mg, 175 mg, 200 mg, 250 mg, 300 mg, 350 mg, 400 mg, 450 mg, 500 mg, 600 mg, 700 mg, 800 mg, 900 mg or 1000 mg. Particular ranges include about 50 mg to about 500 mg, or from about 100 mg to about 200 mg.

The balance of ingredients in the sustained-release active ingredient pharmaceutical formulation can be chosen, for example, from modified polysaccharides such as, for example, methylcellulose (MC) and microcrystalline cellulose (MCC). In some embodiments, the formulation comprises between about 3% to about 99.9% microcrystalline cellulose by weight. In certain embodiments, the formulation comprises about 3% MCC. In other embodiments, the formulation comprises about 5% MCC. In further embodiments, the formulation comprises about 10% MCC. In yet other embodiments, the formulation comprises about 30% MCC. In further embodiments, the formulation comprises about 50% MCC.

In some embodiments, the sustained-release pharmaceutical formulation comprises about 0% to about 40% MC. In certain embodiments, the formulation comprises about 3% MC. In other embodiments, the formulation comprises about 5% MC. In further embodiments, the formulation comprises about 10% MC. In yet other embodiments, the formulation comprises about 30% MC. In further embodiments, the formulation comprises about 40% MC. In some embodiments, the formulation comprises about 95% active ingredient and the remaining 5% is divided between MC and MCC.

The dissolution rate of the sustained-release pharmaceutical formulation determines how quickly active ingredient becomes available for absorption into the blood stream and therefore controls the bioavailability of active ingredient. Dissolution rate is dependent on the size and the composition of the dosage form. In some embodiments, the dissolution rate of the formulation can be by changed by altering the additional components of the formulation. Disintegrants, such as starch or corn starch, or crosslinked PVPs, can be used to increase solubility when desired. Solubilizers can also be used to increase the solubility of the formulations. In some embodiments alternative binders, such as hydroxypropylmethyl cellulose (HPMC), hydroxypropyl cellulose (HPC), methyl cellulose (MC), PVP, gums, xanthine, and the like, can be used to increase the dissolution rate.

In some embodiments the dissolution rate of the formulation can be decreased by adding components that make the formulation more hydrophobic. For example, addition of polymers such as ethylcelluloses, wax, magnesium stearate, and the like decreases the dissolution rate.

In some embodiments, the dissolution rate of the sustained-release pharmaceutical formulation is formulated so as to control the plasma levels of active ingredient.

For example, the sustained-release pharmaceutical formulation can be formulated so as to achieve plasma levels of active ingredient that are, for example, at least, or at least about, more than, or more than about, 5 ng/ml, 10 ng/ml, 15 ng/ml, 20 ng/ml, 25 ng/ml, 30 ng/ml, 40 ng/ml, 50 ng/ml, 60 ng/ml, 70 ng/ml, 80 ng/ml, 90 ng/ml, or 100 ng/ml. The sustained-release pharmaceutical formulation can be formulated so as to achieve plasma levels of active ingredient that are, for example, up to, or up to about, less than, or less than about, 25 ng/ml, 30 ng/ml, 40 ng/ml, 50 ng/ml, 60 ng/ml, 70 ng/ml, 80 ng/ml, 90 ng/ml, 100 ng/ml, 125 ng/ml, 150 ng/ml, 175 ng/ml, 200 ng/ml, 250 ng/ml, 300 ng/ml, 350 ng/ml, 400 ng/ml, 450 ng/ml, or 500 ng/ml. Particular ranges are from about 10 ng/ml to about 150 ng/ml, from about 20 ng/ml to about 100 ng/ml, or from about 40 ng/ml to about 80 ng/ml. It is contemplated that such ranges can be favorable for achieving therapeutic levels of active ingredient without causing sufficient inhibition of MAO inhibition in the digestive tract and liver so as to cause the cheese effect.

In some embodiments, the dissolution rate of the sustained-release pharmaceutical formulation is such that about 25% of the active ingredient in the dosage form is dissolved within the first hour, about 60% of the active ingredient is dissolved within the first 6 hours, about 80% of the active ingredient is dissolved within the first 9 hours, and substantially all of the active ingredient is dissolved within the first 12 hours. In other embodiments, the dissolution rate of the sustained-release pharmaceutical formulation is such that about 35% of the active ingredient in the dosage form is dissolved within the first hour, about 85% of the active ingredient is dissolved within the first 6 hours, and substantially all of the active ingredient is dissolved within the first 9 hours. In yet other embodiments, the dissolution rate of the sustained-release pharmaceutical formulation in the dosage form is such that about 45% of the active ingredient in the beads is dissolved within the first hour, and substantially all of the active ingredient is dissolved within the first 6 hours.

The dissolution rate of the formulation can also be slowed by coating the dosage form. Examples of coatings include sustained-release polymers.

The sustained-release pharmaceutical formulation can take about, for example, from 2, 4, 6, or 8 hours to about 15, 20, or 25 hours to dissolve. Preferably, the formulation has a dissolution rate of from about 3, 4, 5, or 6 to about 8, 9, or 10 hours.

Another embodiment provides a method of preparing sustained-release pharmaceutical formulation. The method comprises mixing active ingredient with an excipient and/or filler to form a mixture, and forming a suitable dosage form (e.g., tablet, bead, etc.) from the mixture. In some embodiments, the method of preparing the formulation further comprises adding another excipient and/or filler to the mixture prior to forming the dosage form. The filler and excipient are as described herein. In an embodiment, the active ingredient is mixed with the filler and/or excipient to form a wet mixture. The wet mixture can then be formed into particles or beads, which can then be dried. The dried product can then be tableted or placed into a gelatin capsule for oral delivery.

In an embodiment, the sustained-release pharmaceutical formulation is in the form of beads. In some embodiments, the beads comprise active ingredient and a filler. In other embodiments, the beads further comprise an excipient. In some embodiments, the filler and/or the excipient are in polymeric form.

As used herein, “beads” can be, for example, spheres, pellets, microspheres, particles, microparticles, granules, and the like. The beads can have any desired shape. The shape can be, for example, spherical, substantially spherical, rod-like, cylindrical, oval, elliptical, granular, and the like. The size and shape of the bead can be modified, if desired, to alter dissolution rates. The beads can be coated or can be uncoated. The beads can be formed into a capsule for oral delivery, a tablet, or any other desired solid oral dosage form, with or without other ingredients.

In an embodiment, a pharmaceutical formulation comprises a bead that comprises sustained-release active ingredient and a filler. In some embodiments the bead further comprises an excipient. In some embodiments the filler is a polymer. In some embodiments the excipient is a polymer. In some embodiments the filler is selected from the group consisting of methylcellulose, hydroxypropyl methylcellulose (HPMC), hydroxypropylcellulose (HPC), corn starch, polyvinyl alcohol (PVA), polyvinylpyrrolidone (PVP), and cross-linked PVP. In some embodiments the excipient is selected from the group consisting of acetyltriethyl citrate (ATEC), acetyltri-n-butyl citrate (ATBC), aspartame, lactose, alginates, calcium carbonate, carbopol, carrageenan, cellulose, cellulose acetate phthalate, croscarmellose sodium, crospovidone, dextrose, dibutyl sebacate, ethylcellulose, fructose, gellan gum, glyceryl behenate, guar gum, lactose, lauryl lactate, low-substituted hydroxypropyl cellulose (L-HPC), magnesium stearate, maltodextrin, maltose, mannitol, methylcellulose, microcrystalline cellulose, methacrylate, sodium carboxymethylcellulose, polyvinyl acetate phathalate (PVAP), povidone, shellac, sodium starch glycolate, sorbitol, starch, sucrose, triacetin, triethylcitrate, vegetable based fatty acid, xanthan gum, and xylitol. In some embodiments the bead comprises active ingredient, methylcellulose and microcrystalline cellulose. In some embodiments the bead comprises from about 0.1% to about 95% active ingredient by weight. In some embodiments the bead comprises between about 3% to about 99.9% microcrystalline cellulose by weight. In some embodiments the bead comprises about 0% to about 40% methylcellulose by weight.

Since modifications will be apparent to those of skill in this art, it is intended that this invention be limited only by the scope of the appended claims.

1. A sustained release oral pharmaceutical product comprising a phenoxathiin-based MAO-A inhibitor of the following formula: 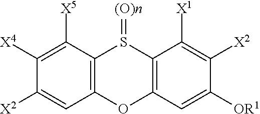
wherein n is 0, 1 or 2; R¹ is a branched or straight chain C1-5 alkyl or C3-6 cycloalkyl optionally substituted with hydroxyl, or one or more halogens; and X¹, X², X³, X⁴, and X⁵ are either all hydrogens or one or two of X¹, X², X³, X⁴, and X⁵ are halogen and the remainder are hydrogens, with the proviso that when n is 0 or 1 and each X is hydrogen, R¹ is not methyl.

2. The sustained release product of claim 1, wherein the phenoxathiin-based MAO-A inhibitor is 3-fluoro-7-(2,2,2-trifluoroethoxy)phenoxathiin 10,10-dioxide.

3. The sustained release product of claim 1, wherein said product is a tablet.

4. The sustained release product of claim 1, wherein said product is a capsule.

5. The sustained release product of claim 1, wherein said product is a core sheathed in an annular body.

6. The sustained release product of any claim 1, wherein said product is formulated so as to achieve plasma levels of phenoxathiin-based MAO-A inhibitor ranging from about 40 ng/ml to about 80 ng/ml.

7. The sustained release product of claim 1, wherein said product is formulated so as to achieve plasma levels of phenoxathiin-based MAO-A inhibitor ranging from about 10 ng/ml to about 150 ng/ml.

8. The sustained release product of claim 1, wherein said product contains 3-fluoro-7-(2,2,2-trifluoroethoxy)phenoxathiin 10,10-dioxide as the sole active ingredient.

9. The sustained release product of claim 2, wherein said 3-fluoro-7-(2,2,2-trifluoroethoxy)phenoxathiin 10,10-dioxide is characterized as having a melting point at about 169-175° C.

10. The sustained release product of claim 2, wherein said 3-fluoro-7-(2,2,2-trifluoroethoxy)phenoxathiin 10,10-dioxide is characterized as being in crystalline form and having an x-ray powder diffraction peak at 2θ=11.0°, using CuK_{α} radiation.

11. The sustained release product of claim 1, wherein said product is a tablet containing about 50 to 500 milligrams of 3-fluoro-7-(2,2,2-trifluoroethoxy)phenoxathiin 10,10-dioxide.

12. An oral pharmaceutical dosage form comprising 3-fluoro-7-(2,2,2-trifluoroethoxy)phenoxathiin 10,10-dioxide and adapted to retard release of 3-fluoro-7-(2,2,2-trifluoroethoxy)phenoxathiin 10,10-dioxide in the digestive tract.

13. The oral pharmaceutical dosage form of claim 12 that is a tablet, a capsule, or a core sheathed in an annular body.

14. The pharmaceutical dosage form of claim 13 that is a tablet.
